(19) United States
(12) Patent Application Publication
(10) Pub. No.: US 2007/0203371 A1
(43) Pub. Date: Aug. 30, 2007

(54) Process for producing polytrimethylene ether glycol

(76) Inventors: Hari Babu Sunkara, Hockessin, DE (US); Howard C. Ng, Kingston (CA); Wei Zheng, Ottawa (CA)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/654,865

(22) Filed: Jan. 18, 2007

Related U.S. Application Data
(60) Provisional application No. 60/761,291, filed on Jan. 23, 2006.

(51) Int. Cl. C07C 31/18

(52) U.S. Cl. 568/866

(57) ABSTRACT

A process for producing polytrimethylene ether glycol by polycondensing 1,3-propanediol using a catalyst comprising an acid and a base, at a temperature of from about 165 to about 175° C.

### CROSS-REFERENCE TO RELATED APPLICATIONS

This application claims priority under 35 U.S.C. § 119 from U.S. Provisional Application Ser. No. 60/761,291 (filed Jan. 23, 2006), the disclosure of which is incorporated by reference herein for all purposes as if fully set forth.

### FIELD OF THE INVENTION

The present invention relates to a process for preparing polytrimethylene ether glycol.

### BACKGROUND OF THE INVENTION

Preparation of polytrimethylene ether glycols, by acid catalyzed polycondensation of 1,3-propanediol is well known in the art.

U.S. Pat. No. 2,520,733 discloses polymers and copolymers of trimethylene glycol and a process for the preparation of these polymers from 1,3-propanediol in the presence of a dehydration catalyst such as iodine, inorganic acids (e.g. sulfuric acid) and organic acids. Polymers of molecular weight from about 100 to about 10,000 are mentioned.

U.S. Pat. No. 6,720,459 and U.S. Pat. No. 6,977,291 disclose processes for preparation of polytrimethylene ether glycol from 1,3-propanediol using a polycondensation catalyst, preferably an acid catalyst.

It is also well known that the polytrimethylene ether glycol produced from the acid catalyzed polycondensation of 1,3-propanediol may have quality problems, in particular, color that is not acceptable for particular applications. The polymerization process conditions and stability of the polymer may be responsible for discoloration to some extent. Polytrimethylene ether glycols are easily discolored by contact with oxygen or air, particularly at elevated temperatures, and so the polymerization is effected under a nitrogen atmosphere and the polyether diols are stored in the presence of inert gas. As an additional precaution, a small concentration of a suitable antioxidant is often added.

Attempts have been made in the past to reduce the color of polytrimethylene ether glycols produced from the above processes by conventional means. For instance, U.S. Pat. No. 2,520,733 notes the peculiar discoloration tendency for the polytrimethylene ether glycol from the polymerization of 1,3-propanediol in the presence of acid catalyst and discloses development of a process for the purification of polyols prepared from 1,3-propanediol in the presence of acid catalyst (2.5 to 6% by weight) and at a temperature from about 175° C. to 200° C. This purification process involves percolation of the polymer through Fuller's earth followed by hydrogenation. This extensive purification process gave a final product that was light yellow in color. In fact, this procedure yielded polytrimethylene ether glycol (Example XI therein) for which the color was only reduced to an 8 Gardner color, which corresponds to an APHA value of >300 and is totally inadequate for current requirements.

US2004/0225162A1 discloses a process for improving the color of polytrimethylene ether glycol comprising contacting polytrimethylene ether glycol having color with adsorbent and separating the polytrimethylene ether glycol and adsorbent, wherein the polytrimethylene ether glycol, after contact with the adsorbent, has a molecular weight of about 250 to about 5000 and a APHA color of less than about 50. US2004/0225163A1 discloses a process for improving the color of polytrimethylene ether glycol comprising contacting the polymer having color with hydrogen in the presence of a hydrogenation catalyst, has a APHA color of less than 50.

Recently, JP-A-2004/182974 and US2005/0272911A1 disclosed an improved process for production of poly(alkylene ether) glycols, in particular polytrimethylene ether glycol, by polycondensation of the corresponding alkylene diol in the presence of a catalyst containing both an acid and a base. The preferred acid is sulfuric acid and the preferred base is pyridine. Polycondensation temperatures are stated to be generally in the range of 120-250° C., and more narrowly in the range of 140-200° C. In the examples presented in JP-A-2004/182974, the polycondensation was described as being carried out at 147-152° C.; the examples presented in US2005/0272911 A1 describe polycondensation at 155° C. +/− 2° C. The product is reported to be of light color and with a high degree of polymerization.

All of the above-identified publications are incorporated by reference herein for all purposes as if fully set forth.

As demonstrated in the examples provided herein, it has been found that, contrary to the results reported in previously incorporated JP-A-2004/182974 and US2005/0272911 A1, at polycondensation temperatures below about 160° C., the base modified acid catalyst does not provide improvement in color and polymerization rate over what is obtainable under the same conditions with acid catalyst alone. Further, it has been found that, when the polycondensation temperature is too high (above about 175° C.), the base modified acid catalyst provides a high reaction rate, but the product color deterioriates to a point that becomes unacceptable.

The present invention described herein relates to a process in which the use of base modified acid catalyst actually provides an improved rate of polymerization, as well as a polytrimethylene ether glycol product with improved color, over what is obtainable under the same conditions with acid catalyst alone.

### SUMMARY OF THE INVENTION

This invention relates to a process for producing polytrimethylene ether glycol comprising: (a) providing 1,3-propanediol and a polycondensation catalyst comprising an acid and a base; and (b) polycondensing the 1,3-propanediol at a temperature of from about 165 to about 175° C. to produce polytrimethylene ether glycol. Preferably, the polycondensation temperature is from about 170 to about 175° C. The polycondensation time is preferably less than about 10 hours, and more preferably less than about 6 hours.

Utilizing the process of the invention, the rate of polymerization of 1,3-propanediol is higher than it is under the same conditions and at the same acid level as compared to no base being used in the polycondensation catalyst. The product polytrimethylene ether glycol has a lower APHA color than that of polytrimethylene ether glycol produced under the same conditions and at the same acid level as compared to no base being used in the polymerization catalyst.

### DETAILED DESCRIPTION OF THE PREFERRED EMBODIMENTS

Unless otherwise defined, all technical and scientific terms used herein have the same meaning as commonly understood by one of ordinary skill in the art to which this invention belongs. In case of conflict, the present specification, including definitions, will control.

Except where expressly noted, trademarks are shown in upper case.

Although methods and materials similar or equivalent to those described herein can be used in the practice or testing of the present invention, suitable methods and materials are described herein.

Unless stated otherwise, all percentages, parts, ratios, etc., are by weight.

When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Use of “a” or “an” are employed to describe elements and components of the invention. This is done merely for convenience and to give a general sense of the invention. This description should be read to include one or at least one and the singular also includes the plural unless it is obvious that it is meant otherwise.

The materials, methods, and examples herein are illustrative only and, except as specifically stated, are not intended to be limiting.

In the context of this disclosure, the general use of the term “1,3-propanediol” is intended to include 1,3-propanediol, 1,3-propanediol dimer and 1,3-propanediol trimer, or mixtures thereof. The term may also be used in the specific context to only refer to 1,3-propane diol.

The 1,3-propanediol employed for preparing the polytrimethylene ether glycols can be obtained by any of the various chemical routes or by biochemical transformation routes. Preferred routes are described in U.S. Pat. No. 5,015,789, U.S. Pat. No. 5,276,201, U.S. Pat. No. 5,284,979, U.S. Pat. No. 5,334,778, U.S. Pat. No. 5,364,984, U.S. Pat. No. 5,364,987, U.S. Pat. No. 5,633,362, U.S. Pat. No. 5,686,276, U.S. Pat. No. 5,821,092, U.S. Pat. No. 5,962,745, U.S. Pat. No. 6,140,543, U.S. Pat. No. 6,232,511, U.S. Pat. No. 6,235,948, U.S. Pat. No. 6,277,289, U.S. Pat. No. 6,297,408, U.S. Pat. No. 6,331,264, U.S. Pat. No. 6,342,646, U.S. Pat. No. 5,633,362, U.S. Pat. No. 5,686,276, U.S. Pat. No. 5,821,092, US2004/0225161A1, US2004/0260125A1 and US2004/0225162A1, the disclosures of which are incorporated by reference herein for all purposes as if fully set forth. A particularly preferred 1,3-propanediol is prepared by a fermentation process using a renewable biological source, such as described in US2005/0069997A1, the disclosure of which is incorporated by reference herein for all purposes as if fully set forth. Preferably the 1,3-propanediol used as the reactant or as a component of the reactant will have a purity of greater than about 99% by weight as determined by gas chromatographic analysis.

As an example of a 1,3-propanediol (PDO) starting material from a renewable source, biochemical routes to 1,3-propanediol have been described that utilize feedstock's produced from biological and renewable resources such as corn feed stock. For example, bacterial strains able to convert glycerol into 1,3-propanediol are found in e.g., in the species Klebsiella, Citrobacter, Clostridium, and Lactobacillus. The technique is disclosed in several patents, including previously incorporated U.S. Pat. No. 5,633,362, U.S. Pat. No. 5,686,276 and U.S. Pat. No. 5,821,092. In previously incorporated U.S. Pat. No. 5,821,092 is disclosed, inter alia, a process for the biological production of 1,3-propanediol from glycerol using recombinant organisms. The process incorporates E. coli bacteria, transformed with a heterologous pdu diol dehydratase gene, having specificity for 1,2-propanediol. The transformed E. coli is grown in the presence of glycerol as a carbon source and 1,3-propanediol is isolated from the growth media. Since both bacteria and yeasts can convert glucose (e.g., corn sugar) or other carbohydrates to glycerol, the process of the invention provided a rapid, inexpensive and environmentally responsible source of 1,3-propanediol monomer.

Preferred starting materials for the process are reactant comprising at least one of 1,3-propanediol, 1,3-propanediol dimer and 1,3-propanediol trimer, or mixtures thereof. Although any of 1 ,3-propanediol, and dimers or trimers of 1,3-propanediol can be used as the reactant in the process of the invention, it is preferred that the reactant comprise about 90% or more by weight of 1,3-propanediol. More preferably the reactant will comprise 99% or more by weight of 1,3-propanediol.

The starting material for the present invention may also contain small amounts, preferably no more than about 20%, more preferably no more than about 10%, by weight of the starting material, of comonomer diols in addition to the reactant 1,3-propanediol or its dimers and trimers without detracting from the efficacy of the process. Preferably, these comonomer diols are aliphatic diols other than 1,3-propanediol. Examples of typical aliphatic diols other than 1,3-propanediol from which polyalkylene ether repeating units may be derived include those derived from aliphatic diols, for example ethylene glycol, 1,6-hexanediol, 1,7-heptanediol, 1,8-octanediol, 1,9-nonanediol, 1,10-decanediol, 1,12-dodecanediol, 3,3,4,4,5,5-hexafluoro-1,5-pentanediol, 2,2,3,3,4,4,5,5-octafluoro-1,6-hexanediol, and 3,3,4,4,5,5,6,6,7,7,8,8,9,9,10,10-hexadecafluoro-1,12-dodecanediol, cycloaliphatic diols, for example 1,4 cyclohexanediol, 1,4-cyclohexanedimethanol and isosorbide, A preferred group of aliphatic diols is selected from the group consisting of ethylene glycol, 2-methyl-1,3-propanediol, 2,2-dimethyl-1,3-propanediol, 2,2-diethyl-1,3-propanediol, 2-ethyl-2-(hydroxymethyl)-1,3-propanediol, 1,6-hexanediol, 1,8-octanediol, 1,10-decanediol, isosorbide, and mixtures thereof. More preferred diols other than 1,3-propanediol are ethylene glycol, 1,6-hexanediol and 1,10-decanediol. A still more preferred comonomer diol is ethylene glycol. Poly(trimethylene-ethylene ether) glycols prepared from 1,3-propanediol and ethylene glycol are described in US2004/0030095A1, the disclosure of which is incorporated by reference herein for all purposes as if fully set forth. Thermal stabilizers, antioxidants and coloring materials may be added to the polymerization mixture or final product if necessary.

The catalyst for the process of the invention comprises both an acid and a base. With respect to the acid component, any acid catalyst or mixture of acid catalysts suitable for acid catalyzed polycondensations of 1,3-propanediol may be used. Preferred acid polycondensation catalysts are described in previously incorporated U.S. Pat. No. 6,977,291 and U.S. Pat. No. 6,720,459. The acid catalysts are preferably selected from group consisting of Lewis acids, Bronsted acids, super acids, and mixtures thereof, and they include both homogeneous and heterogeneous catalysts. More preferably, the acids are selected from the group consisting of inorganic acids, organic sulfonic acids, heteropolyacids and metal salts. Still more preferably the acid is selected from the group consisting of sulfuric acid, hydriodic acid, fluorosulfonic acid, phosphorous acid, p-toluenesulfonic acid, benzenesulfonic acid, methanesulfonic acid, phosphotungstic acid, trifluoromethanesulfonic acid, phosphomolybdic acid, 1,1,2,2-tetrafluoro-ethanesulfonic acid, and 1,1,1,2,3,3-hexafluoropropanesulfonic acid, bismuth triflate, yttrium triflate, ytterbium triflate, neodymium triflate, lanthanum triflate, scandium triflate and zirconium triflate. The catalyst can also be a heterogeneous catalyst selected from the group consisting of zeolites, fluorinated alumina, acid-treated alumina, heteropolyacids and heteropolyacids supported on zirconia, titania alumina and/or silica. An especially preferred catalyst is sulfuric acid.

Bases for use as a component of the catalyst may be organic or inorganic bases. Preferred inorganic bases are the alkali metal hydroxides, carbonates and bicarbonates, where the alkali metal is preferably lithium, sodium or potassium. Organic bases are preferably amines, more preferably tertiary aliphatic, alicyclic and heterocyclic amines. Examples include, but are not restricted to N-methyl imidazole, 1,5-diazabicyclo[4,3,0]-5-nonene, pyridine, quinoline, triethylamine and tributylamine. Preferably, the base comprises at least one member selected from the group consisting of alkali metal hydroxides, alkali metal carbonates, alkali metal bicarbonates, tertiary aliphatic amines and tertiary heterocyclic amines.

More preferably, the base comprises at least one member selected from the group consisting of N-methyl imidazole, 1,5-diazabicyclo[4,3,0]-5-nonene, pyridine, quinoline, triethylamine and tributylamine. More preferred amines contain a pyridine nucleus such as for example pyridine itself or quinoline. A particularly preferred base is pyridine.

Although the catalyst comprises both acid and base, the equivalent ratio of acid and base should be such that acid is always in excess; in other words, the acid catalyst should be present in a stoichiometric excess (acid equivalents to base equivalents). In the context of this disclosure an equivalent of acid is that amount which will react with 1 mole of potassium hydroxide. An equivalent of base is that amount which will react with the same amount of acid as 1 mole of potassium hydroxide.

The preferred equivalent ratio of base to acid in the polycondensation catalyst is from about 0.01:1 to about 0.9:1. More preferably the ratio is from about 0.05:1 to about 0.5:1.

The polymerization process for preparation of poly(alkylene ether) glycols can be batch, semi-continuous, continuous, etc. A preferred batch process for polytrimethylene ether glycol is described in previously incorporated U.S. Pat. No. 6,977,291. In such a batch process in accordance with the present invention, the polytrimethylene-ether glycol is prepared by a process comprising the steps of: (a) providing (1) reactant, and (2) polycondensation catalyst; and (b) polycondensing the reactants to form a polytrimethylene ether glycol.

A preferred continuous process for preparation of polytrimethylene ether glycol is described in previously incorporated U.S. Pat. No. 6,720,459. In such a continuous process in accordance with the present invention, the polytrimethylene ether glycol is prepared by a continuous process comprising: (a) continuously providing (i) reactant, and (ii) polycondensation catalyst; and (b) continuously polycondensing the reactant to form polytrimethylene ether glycol. Preferably the polycondensing is carried out in two or more reaction stages.

In one preferred continuous process, the polycondensation is carried out in an up-flow co-current column reactor and the reactant, and polytrimethylene ether glycol flow upward co-currently with the flow of gases and vapors, preferably where the reactor has at least 3, more preferably at least 8, and up to 30 stages, more preferably up to 15 stages. The reactant can be fed to the reactor at one or multiple locations. In another preferred embodiment, the polycondensation is carried out in a counter current vertical reactor wherein the reactant and polytrimethylene ether glycol flow in a manner counter-current to the flow of gases and vapors. Preferably this reactor has two or more stages. Preferably the reactant is fed at the top of the reactor.

Generally, catalyst levels for use in the process are such that the acid component is about 0.1 % or more, by weight of the diol reactant, more preferably about 0.25% or more, and preferably used in a concentration of about 20% or less, by weight of the reaction mixture, more preferably 10% or less, even more preferably 5% of less, and most preferably 2.5% or less. Catalyst concentrations can be as high as 20 weight % for heterogeneous catalysts and lower than 5 weight % for soluble catalysts.

The reaction time for either batch or continuous polycondensation will depend on the polymer molecular weight that is desired and the reaction temperature, with longer reaction times producing higher molecular weights. Reaction times will preferably be from about 1, more preferably from about 2 hours, and even more preferably from about 3 hours to about 20 hours, more preferably about 10 hours, and even more preferably about 6 hours.

The number average molecular weight of the polytrimethylene ether glycol prepared by the process of the invention is preferably from about 600 to about 5000, and the APHA color is preferably from about 15 to about 80. In preferred embodiments, polytrimethylene ether glycol with APHA color of about 50 or below and number average molecular weight of at least about 1,700 is prepared using a sulfuric acid/pyridine catalyst and a 5-10 hour reaction time.

The invention is illustrated in the following examples. All parts, percentages, etc., referred to in the examples are by weight unless otherwise indicated.

### EXAMPLES

The 1,3-propanediol utilized in the examples was prepared by biological methods described in previously incorporated US2005/0069997A1, and had a purity of >99.8%.

APHA color values were determined using a COLORQUEST XE SPEC-TROPHOTOMETER.

Molecular weights and level of unsaturation were determined by NMR analysis. Proton NMR distinguishes the protons corresponding to the end groups (CH₂-OH) from that of the middle ether groups (CH₂-O- CH₂) and thus it is possible to calculate the molecular weight by comparing the integral areas of these two peaks.

Procedures:

The general procedure for preparing polytrimethylene ether glycol in the Examples summarized below was as follows:

The desired amount of 1,3-propanediol was added to a reactor followed by the desired amount of catalyst. The mixture of 1,3-propanediol and catalyst was then agitated for 10 minutes while being sparged with nitrogen. The reactants were then heated to the desired temperature and held at that temperature for the indicated time. At the end of this time the reaction mixture was allowed to cool to room temperature and then analyzed for color, molecular weight and vinyl unsaturation.

Mole percents in the tables below were calculated on the basis of the total number of moles of 1,3-propanediol, sulfuric acid and pyridine.

In Examples 1-5, the amount of 1,3-propanediol used was 50 g, sulfuric acid 0.652 g and pyridine 0.053 g. In Comparative Examples 1-5, the amount of 1,3-propanediol used was 50 g, and sulfuric acid 0.652 g.

In Examples 6-9, the amount of 1,3-propanediol used was 50 g, sulfuric acid 1.33 g and pyridine 0.536 g. In Comparative Examples 6-8, the amount of 1,3-propanediol used was 50 g, and sulfuric acid 1.33 g.

The results for Examples 1-5 and Comparative Examples 1-5 are presented in Table 1. The results for Examples 6-9 and Comparative Examples 6-8 are presented in Table 2.

TABLE 1
Polytrimethylene Ether Glycol Produced by Base
Modified Sulfuric Acid Catalyst Sulfuric Acid
Level: 1 Mole %, Reaction Time: 10.5 Hours

| | Rxn. Temp. | Pyridine | Mole. | Color | Unsaturation |
|---|---|---|---|---|---|
| Example | (° C.) | (Mole %) | Wt.(Mₙ) | (APHA) | (Meg/Kg) |
| Comp. 1 | 155 | 0 | 464 | 11 | 8.31 |
| 1 | 155 | 0.1 | 412 | 13 | |
| Comp. 2 | 160 | 0 | 587 | 13 | 10.3 |
| 2 | 160 | 0.1 | 527 | 14 | 12.7 |
| Comp. 3 | 170 | 0 | 1199 | 32 | 17.7 |
| 3 | 170 | 0.1 | 1861 | 17 | 20.0 |
| Comp. 4 | 170 | 0 | 1486 | 51 | 17.8 |
| 4 | 170 | 0.1 | 2080 | 27 | 15.9 |
| Comp. 5 | 198 | 0 | 4969 | Black | 87.7 |
| 5 | 198 | 0.1 | 5752 | Black | 187.8 |

The results in Table 1 show that, at 170° C. with base modified catalyst, the polytrimethylene ether glycol produced had a higher molecular weight (1861 and 2080 versus 1199 and 1486) and a lighter color (17 and 27 versus 32 and 51) as compared to polytrimethylene ether glycol produced in the corresponding control runs in the absence of base.

The results in Table 1 also show that at polymerization temperatures at 160° C. or below the modified catalyst did not provide improvement in color or polymerization rate (i.e. molecular weight increase). At high temperatures, e.g. 198° C., the base modified catalyst provided reaction rate improvement, but the polymer color deteriorated and the polymer was not acceptable.

The amount of unsaturation produced in the presence of the base modified catalyst at temperatures of 170° C. or below was comparable to that observed in the absence of base. At high temperature (198° C.), the amount of unsaturation produced in the presence of base was substantially higher.

TABLE 2
Polytrimethylene Ether Glycol Produced by Base
Modified Sulfuric Acid Catalyst Sulfuric Acid
Level: 2 Mole %, Reaction Time: 5 Hours

| | Rxn. Temp. | Pyridine | Mole. | Color | Unsaturation |
|---|---|---|---|---|---|
| Example | (° C.) | (Mole %) | Wt.(Mₙ) | (APHA) | (Meg/Kg) |
| Comp. 6 | 165 | 0 | 976 | 48 | 17.2 |
| 6 | 165 | 1 | | | |
| Comp. 7 | 170 | 0 | 1505 | 143 | 23.4 |
| 7 | 170 | 1 | 1674 | 20 | 27.6 |
| 8 | 170 | 1 | | 22 | |
| Comp. 8 | 175 | 0 | 2095 | 476 | 24.1 |
| 9 | 175 | 1 | 3261 | 79 | 41 |

The results in Table 2 further demonstrate the effect of the base modified catalyst in reaction rate and color improvement in the optimum temperature range between 165 and 175° C., with the best improvement combining lower color, increased molecular weight and lower vinyl ends group content observed at 170° C.

Examples 10 and 11 and Comparative Examples 9 and 10, the results of which are presented in Table 3, were carried out to determine the effect of reaction time at a reaction temperature of 170 C. In Examples 10 and 11 the amount of 1,3-propanediol used was 50 g, sulfuric acid 1.33 g and pyridine 0.536 g. In Comparative Examples 9 and 10 the amount of 1,3-propanediol used was 50 g, and sulfuric acid 1.33 g.

TABLE 3
Polytrimethylene Ether Glycol Produced by Base Modified
Sulfuric Acid Catalyst Effect of Reaction Time at
170° C., Sulfuric Acid Level: 2 Mole %

| | Time | Pyridine | Mole. | Color | Unsaturation |
|---|---|---|---|---|---|
| Example | (Hours) | (Mole %) | Wt.(Mₙ) | (APHA) | (Meg/Kg) |
| Comp. 9 | 5 | 0 | 1505 | 143 | 23.4 |
| 10 | 5 | 1 | 1674 | 20 | 27.6 |
| Comp. 10 | 10.5 | 0 | 2491 | 1388 | 26.2 |
| 11 | 10 | 1 | 4608 | 1425 | 15.4 |

The results in Table 3 show that at longer reaction times using the base modified catalyst the reaction rate improved (as determined by molecular weight), but the polymer color deteriorated. These results demonstrate that the improvement provided by the modified catalyst is dependent not only on the reaction temperature but also on the polymerization time, with a reaction time less than about 10 hours being preferred and less than about 6 hours most preferred.

In general, the results reported above indicate that process of the invention for preparing polytrimethylene ether glycol has at least two advantages over the similar process utilizing acid polycondensation catalyst but no base. First, higher molecular weight polytrimethylene ether glycol was produced in the presence of base than in its absence in the same reaction time, indicating a higher polymerization (reaction) rate in the presence of base. Second, APHA color improvement was 100% or better when base was used over what was obtained with no base at the same acid concentration and reaction time.

The foregoing disclosure of embodiments of the present invention has been presented for purposes of illustration and description. It is not intended to be exhaustive or to limit the invention to the precise forms disclosed. Many variations and modifications of the embodiments described herein will be obvious to one of ordinary skill in the art in light of the disclosure.

What is claimed is:

1. A process for producing polytrimethylene ether glycol comprising:
(a) providing 1,3-propanediol and a polycondensation catalyst comprising an acid and a base;
(b) polycondensing the 1,3-propanediol at a temperature of from about 165 to about 175° C. to produce polytrimethylene ether glycol.

2. The process of claim 1, wherein the temperature is from about 170 to about 175° C.

3. The process of claim 1, wherein the acid comprises at least one member of the group consisting of sulfuric acid, phosphoric acid, hydriodic acid, fluorosulfonic acid, heteropolyacids, p-toluenesulfonic acid, benzenesulfonic acid, methanesulfonic acid, trifluoromethanesulfonic acid, 1,1,2,2-tetrafluoroethanesulfonic acid and 1,1,1,2,3,3-hexafluoropropanesulfonic acid.

4. The process of claim 1, wherein the acid comprises sulfuric acid.

5. The process of claim 1, wherein the base comprises at least one member of the group consisting of alkali metal hydroxides, alkali metal carbonates, alkali metal bicarbonates, tertiary aliphatic amines and tertiary heterocyclic amines.

6. The process of claim 5, wherein the base comprises at least one member of the group consisting of N-methyl imidazole, 1,5-diazabicyclo[4,3,0]-5-nonene, pyridine, quinoline, triethylamine and tributylamine.

7. The process of claim 1, wherein the base comprises pyridine.

8. The process of claim 1, wherein the equivalent ratio of base to acid is from about 0.01:1 to about 0.9:1.

9. The process of claim 1, wherein the equivalent ratio of base to acid is from about 0.05:1 to about 0.5:1.

10. The process of claim 1, wherein the 1,3-propanediol is derived from a fermentation process using a renewable biological source.

11. The process of claim 1, wherein the acid comprises sulfuric acid and the base comprises pyridine.

12. The process of claim 1, wherein the 1,3-propanediol is polycondensed to a polytrimethylene ether glycol having a number average molecular weight of from about 600 to about 3,000.

13. The process of claim 1, wherein the 1,3-propanediol is polycondensed to a polytrimethylene ether glycol having an APHA color of from about 15 to about 80.

14. The process of claim 1, wherein the polycondensation time is less than about 10 hours.

15. The process of claim 1, wherein the polycondensation time is less than about 6 hours.
